(19) United States
(12) Patent Application Publication
(10) Pub. No.: US 2018/0303998 A1
(43) Pub. Date: Oct. 25, 2018

(54) Novel Treatment Method for Cockayne Syndrome

(71) Applicant: Marv Enterprises, LLC, Hermitage, PA (US)

(72) Inventor: Mitchell S. Felder, Hermitage, PA (US)

(21) Appl. No.: 15/933,619

(22) Filed: Mar. 23, 2018

Related U.S. Application Data
(60) Provisional application No. 62/488,649, filed on Apr. 21, 2017.

(51) Int. Cl. A61M 1/36; B01D 61/24; A61K 39/395; A61K 47/64

(52) U.S. Cl. CPC A61M 1/362; A61M 1/3615; A61M 1/3633; A61M 1/3687; B01D 61/243; A61M 2205/75; A61K 47/643; A61M 2202/0445; A61M 2202/0021; A61M 2202/0464; A61K 39/3955

(57) ABSTRACT

Disclosed is a method for treating of Cockayne Syndrome (CS). Specifically, the invention pertains to a method for the extracorporeal treatment of a body fluid by removing the body fluid from a living body diseased with CS, and applying a targeted antibody conjugated to a moiety to at least one Cockayne Syndrome antigen in the body fluid, creating an antibody-antigen moiety complex, removing antibody-antigen moiety complex from the body fluid, and returning the purified body fluid to the body.

This application claims priority to U.S. Provisional Application Ser. No. 62/488,649 filed on Apr. 21, 2017, the contents of which are fully incorporated in this disclosure.

### FIELD OF THE INVENTION

This disclosure relates to a treatment for slowing and reversing the disease processes relating to Cockayne Syndrome utilizing an extracorporeal methodology to achieve this purpose.

### BACKGROUND OF THE INVENTION

Cockayne syndrome (CS) is an inherited autosomal recessive trait where both parents are obligate carriers of an abnormal gene (ERCC6 or ERCC8). The incidence of CS is estimated at approximately 1 per 2.7 per million births, although according to recent studies, CS is likely under diagnosed. Children with CS typically experience abnormalities that aid in the differential diagnosis of the disease. These abnormalities include congenital anomalies of the face, limbs, heart, viscera, metabolic and neurologic crises, hematologic issues and a variety of malignancies. CS spans a phenotypic spectrum that includes CS type I, the “classic” or “moderate” form, CS type II, a more severe form with symptoms present at birth, which overlaps with cerebro-oculo-facio-skeletal syndrome (COFS) or Pena-Shokeir syndrome type II, and CS type III, a milder form, xeroderma pigmentosum-Cockayne syndrome (XP-CS) caused in part by mutations in ERCC1 and ERCC4.

CS type I (moderate CS) is characterized by normal prenatal growth with the onset of growth and developmental abnormalities in the first two years. By the time the disease has become fully manifest, height, weight, and head circumference are far below the norm expected in a healthy child. Progressive impairment of vision, hearing, and central and peripheral nervous system function leads to severe disability; death typically occurs in the first or second decade. A recent study demonstrated that more than 30 proteins are involved in the CS-B interactome and may be involved in the pleiotropic functions of CS-B. CS-B is known to be involved in DNA break repair and checkpoint regulation.

CS type II (severe CS or early-onset CS) is characterized by growth failure at birth, with little or no postnatal neurologic development and is caused by deficits in the CS-A gene. Congenital cataracts or other structural anomalies of the eye may be present. Affected children have early postnatal contractures of the spine (kyphosis, scoliosis) and joints. Death usually occurs by age seven years.

CS type III (mild CS or late-onset CS) is characterized by essentially normal growth and cognitive development, or by late onset development of the syndrome. Xeroderma pigmentosum-Cockayne syndrome (XP-CS) includes facial freckling and early skin cancers typical of XP and some features typical of CS, including intellectual disability, spasticity, short stature, and hypogonadism. XP-CS does not include skeletal involvement, the facial phenotype commonly associated with CS, or CNS dysmyelination and calcifications.

Classic CS is diagnosed by clinical findings including postnatal growth failure and progressive neurologic dysfunction along with other minor criteria. Molecular genetic testing or a specific DNA repair assay on fibroblasts can confirm the diagnosis. The two primary genes in which mutations are known to cause CS are ERCC6, which codes for the CS-B protein (65%) of and ERCC8, which codes for the CS-A protein (35%). Most variants are identified by sequence analysis of ERCC6 and ERCC8. Both genes are members of the family of excision-repair cross complementation group. Mutation in either of these genes results in deficits in DNA checkpoint regulation and in DNA repair mechanisms. Recent evidence also suggests a role for CS-A and CS-B in response to oxidative stress. Currently, the only drug therapy for patients suffering from CS is Prodarsan. Prodarsan is a drug (10% D-mannitol) that was provided orphan status and has been evaluated for its ability to reduce the accumulation of DNA damage. Thus, this application represents a novel method for meaningful therapy for those affected by CS.

### SUMMARY OF THE INVENTION

Disclosed is a method of extracorporeal treating a Cockayne Syndrome patient's body fluid: for example, their blood, or cerebrospinal fluid (CSF). U.S. Ser. No. 13/128,870, U.S. Ser. No. 13/128,177, U.S. Ser. No. 13/254,855, U.S. 61/612,474, and U.S. 61/644,292 are hereby incorporated by reference. Treating a Cockayne Syndrome patient comprises removing the body fluid from a patient, applying an extracorporeal treatment to the body fluid, removing the extracorporeal treatment, identifying any remaining extracorporeal treatment, and returning the body fluid to the patient.

### DETAILED DESCRIPTION OF THE INVENTION

The method disclosed comprises treating a patient's body fluid extracorporeally with an antibody or multiple antibodies designed to react with targeted antigens that are associated with Cockayne Syndrome (Cockayne Syndrome Targeted Antigens-CSTA). For the purposes of this disclosure and unless stated specifically otherwise, CSTAs include but are not limited to: death-associated protein 1 (DAP1), calpain, p21, mammalian target of rapamycin (mTOR), insulin growth factor-1 (IGF-1), lipofuscin, p16 (p16INK4a), cyclin-dependent Kinase inhibitor 2A (CDKN2A), Senescence associated β-gal (SA-β-gal), promyelocytic leukemia (PML) protein, transforming growth factor β (TGF-β), interleukin-6 (IL-6), indoleamine 2,3-dioxygenase, soluble tumor necrosis factor-receptor 55 (sTNF-R55), soluble tumor necrosis factor-receptor 75 (sTNF-R75), Progerin, oxygen and nitrogen based free radicals: (superoxide, nitric oxide, hydroxyl radical, perioxynitrite, nitrosoperoxycarbonate, hydrogen peroxide, hypochlorite); malondialdehyde (MDA, propanedial); tumor necrosis factor-α (TNF-α), and mitogen activated protein kinases (MAPK). Antibodies that target CSTAs are referred to as CSTA targeted antibodies. Preferably, the quantity of CSTA targeted antibodies used binds substantially all of the CSTA present in the body fluid. The CSTA targeted antibodies used to treat the body fluid can be targeted to only one CSTA, or the CSTA targeted antibodies can be targeted to more than one CSTA.

The CSTA targeted antibodies can be conjugated with a moiety such as albumin. Conjugation with such a moiety permits efficacious dialysis for the removal of the CSTA targeted antibody-CSTA complex. Removal of the complex can be done using a dialysis machine as known to those with skill in the art. One dialysis modality is continuous renal replacement therapy (CRRT), which can remove large quantities of filterable molecules from the extracorporeal blood. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis. The sieving coefficient (SC) is the ratio of the molecular concentration in the filtrate to the incoming blood. A SC close to zero implies that the moiety-antibody-targeted antigen complex may not pass through the filter. A filtration rate of about 50 mL/min is generally satisfactory. Other methods of increasing the removability of the CSTA targeted antibody-CSTA moiety include the use of temporary acidification of the body fluid extracorporeally using organic acids to compete with protein binding sites.

Alternatively, removal can be done using a molecular filter. For example, molecular adsorbents recirculating system (MARS), which may be compatible and/or synergistic with dialysis equipment. MARS technology can be used to remove small to average sized molecules. Artificial liver filtration presently uses this technique.

An alternative embodiment would utilize a “designer” CSTA targeted antibody that is conjugated with a macromolecular moiety instead of an albumin moiety to form a complex with the CSTA. The CSTA targeted antibody-moiety-CSTA complex can then be removed based on molecular size, protein binding, solubility, chemical reactivity, and combinations thereof. For example, the macromolecular moiety can be 1.000 mm to 0.00001 mm in diameter and the body fluid can flow through a series of micro screens or size exclusion membrane which contain openings with a diameter 50% to 99.99999% less than the diameter of the designer CSTA target antibody-macromolecular moiety and therefore captures the CSTA targeted antibody-moiety-CSTA complex. The opening(s) must have a diameter of at least 25 microns to allow for the passage of the non-pathologic inducing body fluid constituents, that will ultimately be returned to the patient. Other examples for filtering the body fluid to remove the CSTA targeted antibody-moiety-CSTA complexes, can include a molecular sieve, such as zeolite, or porous membranes that capture complexes comprising molecules above a certain size, such membranes can comprise polyacrylonitrile, polysulfone, polyamides, cellulose, cellulose acetate, polyacrylates, polymethylmethacrylates, and combinations thereof.

Alternatively, immunoaffinity chromatography may be used in which the heterogeneous group of molecules in the body fluid will undergo a purification process. There will be an entrapment on a solid or stationary phase or medium. Only the targeted antigens will be trapped using immunoaffinity chromatography. A solid medium can be removed from the mixture, washed and the CSTA(s) may then be released from the entrapment through elution.

Alternatively, gel filtration chromatography may be utilized in which the body fluid is used to transport the sample through a size exclusion column that will be used to separate the target antigen(s) by size and molecular weight.

In another embodiment, a designer antibody containing an iron (Fe) moiety that targets CSTAs can be used. A Fe-CSTA targeted antibody-CSTA complex is then created. This iron-containing complex may then be efficaciously removed from the body fluid using a strong, localized magnetic force field.

After the body fluid had passed through a removal process, a portion the body fluid can be tested to identify any remaining unbound CSTAs or CSTA targeted antibody complexes before returning the body fluid to the patient. Body fluids with unacceptably large concentrations of unbound CSTAs or the CSTA targeted antibody complexes remaining can then be put back through the removal process(es) before returning the body fluid to the patient. CSTAs can be identified using standard enzyme-linked immunosorbent assay (ELISA) methodology. ELISA is a biochemical technique, which allows for the detection of an antigen in a sample. In ELISA an antigen is affixed to a surface, and then an antibody is utilized for binding to the antigen. The antibody is linked to an enzyme, which enables a color change in the substrate.

Other strategies may be used to validate the level of unbound CSTAs or CSTA targeted antibody complexes in the body fluid: Western blotting technology, UV/vis spectroscopy, mass spectroscopy, chromatography, and surface plasmon spectroscopy.

Another alternative methodology for identifying unbound CSTAs or CSTA targeted antibody complexes remaining in the body fluid would be to utilize molecular weight cut-off filtration. Molecular weight cut-off filtration refers to the molecular weight at which at least 80% of the target antigen(s) is prohibited from membrane diffusion.

In addition to the CSTA targeted antibodies discussed above, the body fluid can also be treated with antibodies target against several microRNAs that are linked to inflammation such as miR222 and miR223. These antibodies can also be conjugated as in the methods described above, complexes that are formed are then removed using the methods described above, and any residual amounts of complexes remaining tested for as described above.

After the removal of the complexes, the cleansed body fluid can then be returned to the patient. For example, a treatment of a patient's blood comprises removing 25 mL to 500 mL of blood from a patient using a catheter, and then applying the treatment to the blood before returning it to the patient via the same catheter. The frequency of such treatments would depend upon an analysis of the underlying symptomatology and pathology of the patient.

The extracorporeal treatment may not be limited to removing CSTA, additional therapeutic agents may be added into the body fluid after the harmful CSTA proteins are removed. These therapeutic agents include, but are not limited to: Lithium chloride, rapamycin, rotenone, p62 (nucleoporin 62), magnesium and coenzyme Q10, recombinant IL-10, sildenafil, amitriptyline, recombinant CS-A, recombinant CS-B, Prodarsan, calcium blockers such as diltiazem, low dose prednisolone and/or deflazacort, Vitamin D3, Vitamin C, and Vitamin E.

Autophagy agonists may also be added to the body fluid: spermidine, fluspirilene, reservatrol, and small molecule enhancers of autophagy (SMER10, SMER18, and SMER28) are non-limited examples. Autophagy inducers may also be added to the body fluid: loperamide, amiodarone, niguldipine, pimozide, nicardipine, and trifluoroperazine are non-limiting examples.

In another embodiment, viral vectors containing one or more genes that encode for proteins associated with Cockayne Syndrome such as ERCC1, ERCC4, ERCC5, ERCC6, ERCC8 or KIAA1530 can be added during the treatment of the body fluid. These vectors can take the form of a variety of replication defective viruses including adenovirus associated virus (AAV), herpes, influenza and/or Coxsackievirus B is used as a vector for the introduction of ERCC6 and/or ERCC8. Significant research has been performed previously with AAV that has shown promise. Live attenuated influenza virus and Coxsackievirus B may also be utilized for the transference of the recombinant KIAA1530, ERCC1, ERCC4, ERCC5, ERCC6 or ERCC8 genes. The methodology of transference utilizing attenuated influenza virus and Coxsackievirus B may be further refined by the insertion of miRNA to better control the level and location of virus replication to affected organ systems in Cockayne Syndrome. Another possible approach for gene targeting is the molecule pullulan. Pullulan is a water-soluble exo-polysaccharide that is produced by yeast like fungus Aureobasidium pullulans. The unique structure of pullulan allows for it to be used as a unique drug delivery platform. Nucleic acid therapeutics can also be employed via the addition of liposomal particles that contain nucleic acids for delivery to the affected tissues. There is a plethora of novel cholesterol-based cationic lipids that can be used for efficient gene delivery.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A method for the extracorporeal treatment of a body fluid carrying at least one Cockayne Syndrome Targeted Antigen (CSTA) comprising:
treating the body fluid carrying at least one unbound CSTA with at least one CSTA targeted antibody in an amount sufficient to bind all the at least one unbound CSTA forming CSTA target antibody-CSTA complexes, wherein the at least one CSTA targeted antibody is conjugated with a moiety;
removing the CSTA targeted antibody-CSTA complexes from the body fluid; and
identifying any remaining CSTA targeted antibody-CSTA complexes.

2. The method of claim 1 wherein the CSTA is at least one member selected from the group consisting of:
death-associated protein 1 (DAP1), calpain, p21, mammalian target of rapamycin (mTOR), insulin growth factor-1 (IGF-1), lipofuscin, p16 (p16INK4a), cyclin-dependent kinase inhibitor 2A (CDKN2A), Senescence associated β-gal (SA-β-gal), promyelocytic leukemia (PML) protein, transforming growth factor β (TGF-β), interleukin-6 (IL-6), indoleamine 2,3-dioxygenase, soluble tumor necrosis factor-receptor 55 (sTNF-R55), soluble tumor necrosis factor-receptor 75 (sTNF-R75), Progerin, oxygen and nitrogen based free radicals: (superoxide, nitric oxide, hydroxyl radical, perioxynitrite, nitrosoperoxycarbonate, hydrogen peroxide, hypochlorite); malondialdehyde (MDA, propanedial); tumor necrosis factor-α (TNF-α), and mitogen activated protein kinases (MAPK).

3. The method of claim 1 wherein the moiety is albumin.

4. The method of claim 1 wherein the moiety is a macromolecule; the macromolecule having diameter ranging from 0.00001 mm-1.00 mm.

5. The method of claim 1 wherein the moiety is iron.

6. The method of claim 1 wherein removing the CSTA targeted antibody-CSTA complexes from the body fluid is done using MARS.

7. The method of claim 1 wherein removing the CSTA targeted antibody-CSTA complexes from the body fluid is done using micro screens.

8. The method of claim 1 wherein removing the CSTA targeted antibody-CSTA complexes from the body fluid is done using continuous renal replacement therapy.

9. The method of claim 1 wherein removing the CSTA targeted antibody-CSTA complexes from the body fluid is done using zeolite.

10. The method of claim 1 wherein removing the CSTA targeted antibody-CSTA complexes from the body fluid is done using membranes that filter for specific molecular sizes.

11. The method of claim 1 wherein removing the CSTA targeted antibody-CSTA complexes from the body fluid is done using immunoaffinity chromatography.

12. The method of claim 1 wherein removing the CSTA targeted antibody-CSTA complexes from the body fluid is done using gel filtration chromatography.

13. The method of claim 1 wherein identifying any remaining CSTA targeted antibody-CSTA complexes is done by enzyme-linked immunosorbent assay (ELISA).

14. The method of claim 1 wherein identifying any remaining CSTA targeted antibody-CSTA complexes is done by molecular weight cut-off filtration.

15. The method of claim 1 further comprised of adding one or more therapeutic agents to the body fluid, wherein the one or more therapeutic agents are at least one member selected from the group consisting of:
recombinant ERCC1, recombinant ERCC4, recombinant ERCC5, recombinant ERCC8, recombinant ERCC6, and recombinant KIAA1530.

16. The method of claim 1 further comprised of adding one or more therapeutic agents to the body fluid, wherein the one or more therapeutic agents are at least one member selected from the group consisting of: Lithium chloride, rapamycin, rotenone, p62 (nucleoporin 62), magnesium and coenzyme Q10, recombinant IL-10, sildenafil, amitriptyline, recombinant CS-A, recombinant CS-B, Prodarsan, calcium blockers such as diltiazem, low dose prednisolone and/or deflazacort, Vitamin D3, Vitamin C, and Vitamin E.

17. The method of claim 1 further comprised of adding one or more therapeutic agents to the body fluid, wherein the therapeutic agents are autophagy agonists.

18. The method of claim 1 further comprised of adding or more therapeutic agents to the body fluid, wherein the therapeutic agents are autophagy inducers.

19. The method of claim 1 further comprising treating the body fluid with antibodies targeted against miR222 and miR223.
